(19) United States
(12) Patent Application Publication
(10) Pub. No.: US 2004/0115708 A1
(43) Pub. Date: Jun. 17, 2004

(54) Method of screening agents for the treatment and prevention of cancer and cachexia and the new use of specific agents for the treatment and prevention of cancer and cachexia

(76) Inventor: C. Murray Ardies, Chicago, IL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 10/665,221

(22) Filed: Sep. 17, 2003

Related U.S. Application Data
(60) Provisional application No. 60/421,679, filed on Oct. 28, 2002.

(51) Int. Cl. C12Q 1/68; G01N 33/574

(52) U.S. Cl. 435/6; 435/7.23

(57) ABSTRACT

A method for screening agents to determine possible use as treatment and prevention drugs is provided. More specifically, cells, living organisms, mammals, or tissue are treated with the compound or agent of interest and then measured for transient activation of stress-response pathways. Activation of any of the genes or signaling molecules associated with a stress-response pathway is then determined. A transient activation of any of the genes within these pathways of transcription activation indicates the agent is a candidate for a drug, or a source of a drug for the treatment and prevention of cachexia, other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities. Methods of treatment and uses of specific agents are also provided.

### TECHNICAL FIELD

The present invention is directed to a method of screening chemical compounds, or agents, for potential use as drugs. More particularly, the present invention is directed to a method for screening of agents for potential use in the treatment and prevention of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities.

The present invention also is directed to the new use of the following specific agents: diacylglycerol or any of its' derivatives or analogues; phospholipids, including but not limited to phosphatidylinositol or any of its' derivatives or analogues; hydrogen peroxide and any other lipid (including but not limited to short-, medium-, or long-chain fatty acid peroxides) or water soluble peroxide and peroxide derivative which retains any of the reactive properties of the original peroxide; oxygen radical generating system capable of generating superoxide anions, hydrogen peroxides, hydroxyl radicals or any other chemical oxidant; calcium; calcium ionophores such as A23187 or thapsigargin and their derivatives. The new use or uses of these specific agents is directed toward the treatment and prevention of cachexia, other wasting disorders, cancer, atherosclerosis, heart disease, acute or chronic autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities.

### BACKGROUND OF THE INVENTION

There is strong evidence that high endogenous expression and activity of the various protective antioxidant and phase H enzymes are important for protecting the body from disorders caused or mediated by chronic oxygen radical damage or chronic chemical toxicities, such as cancer, cachexia and other wasting disorders, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, and Alzheimer's disease. There also is strong evidence that enhancing activity or expression of any factor or factors related to the GLUT4 translocation or GLUT4 activation pathways will in turn enhance insulin sensitivity. There also is strong evidence that enhancing activation or expression of any factors related to or part of the growth hormone signal transduction pathway will enhance endogenous protein synthesis. It is desirable to use such knowledge to create drugs and agents for the treatment and prevention of such disorders.

A major problem in the process of drug discovery, however, is the huge amount of time and expense of identifying candidate agents. There is currently no rapid and easy method to identify possible candidates for the treatment and prevention of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities.

One object of the present invention is to provide a less time consuming and expensive method for identifying such agents.

Another object of the present invention is to provide methods of treatment and prevention and new uses of specific agents for treating these diseases and conditions.

### SUMMARY OF THE INVENTION

One embodiment of the present invention is directed to a method of screening agents as candidates for the treatment and prevention of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities. In this method, a determination of a transient activation of a signaling pathway indicates the agent is a candidate agent or a source of a candidate agent for the treatment or prevention of the above listed conditions.

More specifically, this embodiment is directed to a method of screening agents or sources of agents to determine which are useful for the treatment or prevention of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, or any other condition caused or mediated by chronic oxygen radical damage or by chronic chemical toxicities.

In one alternative of this embodiment, the method includes the steps of (a) treating cells from a living organism with a putative agent (or source of agent) and (b) measuring or determining transient activation of a stress-response pathway. Preferably, the treated cells are mammalian cells, including but not limited to cells from any cell line, human cells and primate cells. Other possible organisms include for example, yeast. The cells are sampled at 10 to 60 minute timed intervals to determine if activation has occurred. Transient activation of a stress-response pathway indicates the tested putative agent or source of agent is useful for prevention or treatment of these wasting disorders.

Preferably, such stress-response pathways include, for example, those of gene products or signaling molecules associated with the mitogen activated protein kinase (MAPK) pathways of transcription activation, stress activated protein kinase (SAPK; a.k.a. JNK-MAPK or c-Jun NH₂ terminal kinase pathway) pathways of transcription activation, hypoxia inducible factor (HIF) pathways of transcription activation, nuclear factor kappa-beta (Nf-κβ) pathways of transcription activation, or the insulin-associated GLUT4 activation or translocation pathways. Activation of a stress-response pathway can be measured by any appropriate methodology. Examples of such methodology include western blot, dot-blot, ELISA, immunocytochemistry, immunohistochemistry, mass-spectroscopy, multiplexed proteomics technology, antibody microarray, or truncated protein analysis to determine enhanced content of gene product(s) in cells, nuclei, or enhanced appearance of gene products within nuclear-protein extracts; phosphorylation-based assays to determine phosphorylation state of any of the gene products, phospholipids, diacylglycerol products or other signaling molecules in said gene or stress-response pathways; gel-shift assays to determine enhanced sequence-specific DNA-binding of the various genes involved in the above-mentioned pathways (such as, but not limited to, AP-1); any reporter-gene assays to determine gene activation; any RT-PCR-based assays (including real-time RT-PCR, in situ hybridization, quantitative and semi-quantitative RT-PCR, and competitive quantitative RT-PCR) to quantify content of mRNA coding for the gene product(s) in question; or any of the RNA- or protein-array or microarray methodologies available to determine gene activation.

Alternatively to treating cells, the method of this embodiment can comprise the step of (a) treating living organisms, living mammals or cultured tissues from mammals of any type, including but not limited to humans and primates, with a putative agent or source of agent.

The method of either alternatives of this embodiment can further comprise the step of a transient activation of any of the genes or signaling molecules within these pathways.

For the purpose of this application, transient activation of the stress-response pathways is defined as: a) an activation of any of the signaling processes which may result in increased synthesis of any of the gene products associated with the stress-response pathways, b) increased nuclear content of any of the gene products associated with the stress-response pathways, or c) increased cellular content of any of the gene products associated with the stress-response pathways, effects which last for more than approximately 1 minute but are no longer apparent within approximately 12 hours of the initial activation. The transient activation effects are further defined in this application as occurring not more than three times in one day for each of seven days each week and not less than once each day for each of three non-consecutive days each week. The transient activation of the stress-response pathways by the agent may be produced, for example, through modifying dosing regimens, dosing apparatus, or dosing formulations, or by modifying the pharmacokinetic properties of the agent, or through modifying any other properties of the agent or its' vehicle or its' delivery apparatus by any other technique readily developed by anyone skilled in the art.

Another embodiment of the present invention, in accordance with the above discovery, is directed to a new use of existing agents diacylglycerol or any of its' derivatives or analogues; phospholipids, including but not limited to phosphatidylinositol or any of its' derivatives or analogues; hydrogen peroxide and any other lipid (including but not limited to short-, medium-, or long-chain fatty acid peroxides) or water soluble peroxide and peroxide derivative which retains any of the reactive properties of the original peroxide; oxygen radical generating system capable of generating superoxide anions, hydrogen peroxides, hydroxyl radicals or any other chemical oxidant; calcium; calcium ionophores such as A23187 or thapsigargin and their derivatives as agents to produce the beneficial effects of a more stable (lasting longer than 24 hours) expression and greater endogenous activity of the various protective antioxidant and phase II enzymes which are important in protecting from disorders caused by or mediated by chronic oxygen radical damage or those disorders caused by chronic chemical toxicities; examples of such diseases include cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, and Alzheimer's disease.

In an additional aspect, this embodiment is further directed to the above agents which can directly or indirectly activate in a transient manner any of the gene or signaling pathways (e.g. step (b) in the first embodiment above) and which are drugs for preventing or treating the various disorders listed in the first embodiment above.

In addition, these agents will lead to enhanced rates of endogenous protein synthesis and increased insulin sensitivity, thereby reducing risk for wasting syndromes, or attenuating existing wasting conditions.

In addition, these agents will lead to an attenuated inflammatory response thereby reducing risk for tissue damage and wasting syndromes secondary to chronic inflammatory conditions.

A further embodiment of the present invention is directed to methods of treatment and prevention of the above conditions using the above-identified specific agents.

### BRIEF DESCRIPTION OF THE DRAWINGS

FIG. 1 illustrates the effect of cachexia and physical exercise on the GLUT4 activation pathways.

FIG. 2 illustrates the insulin-mediated activation of the MAPK pathway of transcription activation.

FIG. 3 illustrates the growth hormone (GH)-mediated activation of the MAPK pathway of transcription activation.

FIG. 4 illustrates the interactive effects of physical exercise, insulin, and growth hormone on activation of the SAPK and MAPK pathways of transcription activation.

FIG. 5 illustrates the effect of acute running exercise on Jun content of lung nuclei from rats.

### DETAILED DESCRIPTION OF THE INVENTION AND EMBODIMENTS THEREOF

The present invention is based, in part, on the discovery that the risk-reduction and treatment benefits of exercise are due to a transient activation of signaling molecules or gene products common to the insulin-signaling pathways, growth hormone, MAPK, SAPK, and NFκβ and possibly the HIF pathways. This transient activation of these pathways leads to a more stable (lasting longer than 24 hours) expression and greater endogenous activity of the various protective antioxidant and phase II enzymes which are important in protecting from disorders caused by or mediated by chronic oxygen radical damage or those disorders caused by chronic chemical toxicities. Examples of such diseases include cancer, cachexia, other wasting disorders, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, and Alzheimer's disease. In addition, transient activation of these pathways also leads to enhanced rates of endogenous protein synthesis and increased insulin sensitivity, thereby reducing risk for wasting syndromes or providing a means to treat the same. While others have produced similar gene-activation results in skeletal muscle, it was not perceived that muscle-specific events relate to risk-reduction for, or to the treatment of, human disorders relating to non-muscle tissues. In addition, as described by many others, many of these genes are known oncogenes which, when they are mutated and they gain constitutive activity, are associated with the development of cancer. It was not perceived that a transient activation of these same oncogenes will result in protective effects. With the results discovered in the present invention, Applicant conceived that the transient activation of genes associated with these pathways by exercise in non-muscle tissues is the source of the risk-reduction and/or treatment benefits of exercise.

Based on Applicant's discovery, it is now believed that many prior references, which relate to the prevention of oxygen radical mediated disorders (such as many cancers) by blocking these genes in those who have normal gene function, may be based on a non-physiological model and may be inherently flawed.

More specifically, for many decades, there has been strong evidence that very low risk for developing heart disease, insulin-resistance disorders, or cancer is strongly associated with repeated physical activity whether the repeated physical activity is performed for personal enjoyment, as part of a regular personal health/lifestyle program, or as part of a training regime for recreational or intensive athletic competition. Repeated physical activity also is well known to be very important as part of a treatment paradigm for cardiac disease, insulin resistance disorders, and more recently, for cancer. There is much evidence as discussed herein that transient activation of any one or any combination of the gene pathways mentioned supra is responsible for the observed benefits of exercise due to the extensive “cross-talk” among the various pathways. The intermittent nature of the repeated physical activity is essential for its use as a prophylactic, treatment, or performance-enhancement paradigm and commonly known principals of exercise indicate that the activity must be performed at a moderate to high intensity for a minimum of three days each week with enhanced performance benefits being maximal with the moderate to intense physical activity being repeated no more than two to three times each day, seven days each week. Repetitions more frequent than this lead to declines in performance and ultimately to declines in health due to what is known as “overtraining syndrome”.

It is this intermittent activation of the gene pathways resulting from the intermittent stress that is primarily responsible (though not necessarily the solely) to the present invention of a new screening method for useful drugs for the treatment or prevention of cachexia, other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, or any other condition caused or mediated by chronic oxygen radical damage or by chronic chemical toxicities.

Further, prior to this discovery, Applicant is unaware of art which indicates that intermittent activation of (known oncogene) gene pathways in non-muscle tissues was responsible for the health-risk reduction and therapeutic benefits of exercise. Prior to this discovery, there existed no convenient screening method to rapidly detect candidate agents for the prevention or treatment of disorders caused wholly or in part by chronic oxygen radical exposure or chronic chemical toxicities. Compounds such as calcium ionophores (and their derivatives) which transiently increase cellular calcium and trigger the activation of these gene pathways, hydrogen peroxide or other short lived, soluble pro-oxidants which directly activate these gene pathways, and short-lived diacylglycerol or phosphorylated phosphatidylinositol (and their derivatives) which directly activate these gene pathways all will have, with appropriate protocols of administration, utility in the prevention or treatment of disorders caused wholly or in part by chronic oxygen radical exposure or chronic chemical toxicities such as cancer, heart disease, or cachexia.

Numerous research papers and extensive reviews have been published on the role of physical activity in promoting human health and in the prevention and treatment of heart disease and cancer. Within the art, the term “physical activity” is commonly used to refer to any bodily movement produced by skeletal muscles which results in an energy expenditure that is substantially greater than that at rest and refers to a single session of activity. The term “physical exercise” as defined by these in the art is any physical activity which is performed repeatedly over prolonged periods of time with the intent of improving fitness or health. The terms “physical exercise” and “repeated physical activity” have been used interchangeably throughout the present application.

Immune function, hormonal status, body fat content, antioxidant enzyme activities, and rates of carcinogen inactivation (phase II enzyme activities) all are altered in a beneficial manner by physical exercise and are mechanistically involved in the known cancer-risk reduction. Several authors have even suggested that physical exercise should be recommended for everyone because of the role it appears to play in preventing cancer.

Regarding cancer treatment, the use of physical exercise would be recommended to reduce fatigue and wasting that possibly is associated with cachexia, enhance quality of life, and to reduce risk of further recurrences of cancer. Cachexia is a syndrome that affects approximately 50% of all cancer patients. It is characterized by weakness, fatigue, anorexia, loss of adipose tissue and skeletal muscle, abnormal metabolism, and impaired immune function. Although total parenteral nutrition with amino acid supplementation can partially reverse some of the negative nitrogen balance observed in cachexic patients, nutritional support to counterbalance the anorexia and weight loss has not been very successful in attenuating this syndrome. In contrast to these nutrition-based treatments, physical exercise (based on the known molecular mechanisms associated with cachexia as discussed below) may be capable of attenuating or preventing this condition.

Cachexia is known to affect skeletal muscle metabolism, resulting in enhanced wasting of these tissues. Not only is the rate of proteolysis increased with cachexia, but the metabolism of glucose and amino acids is greatly altered in cancer patients, in part, due to a decrease in insulin sensitivity.

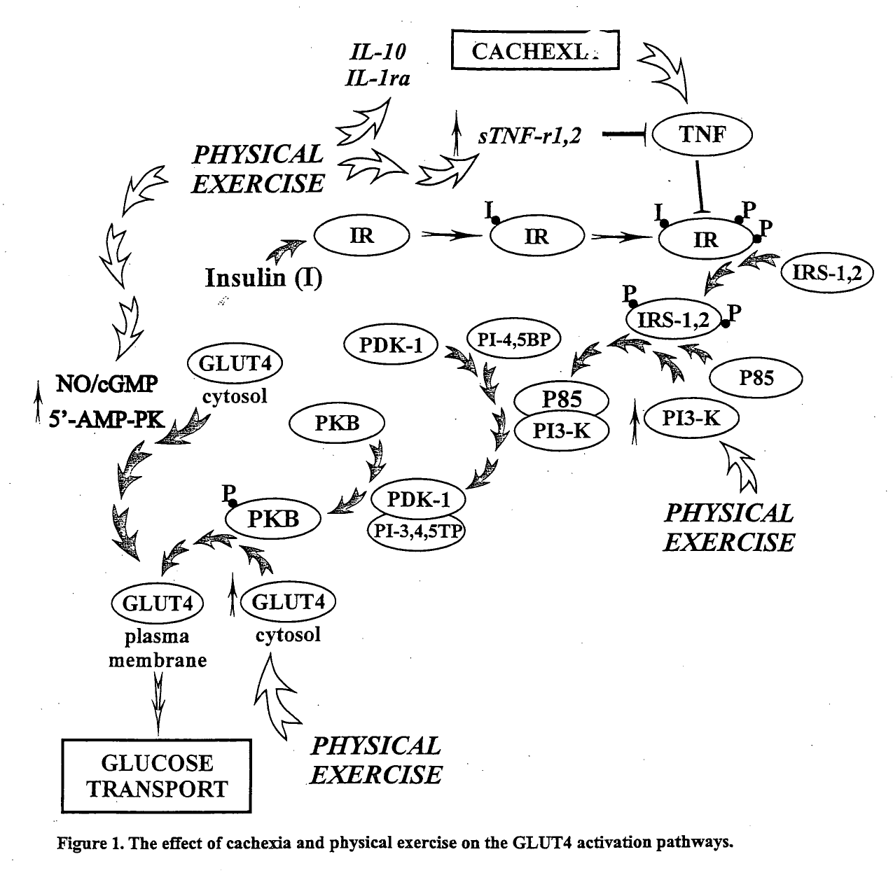

Mechanisms of insulin resistance in cachexia are not well understood; however, because insulin insensitivity and increased TNF levels in serum have been observed with several clinical syndromes, it appears that TNF may be involved. For example, insulin resistance in obese and elderly diabetics is associated with a greater rate of TNF synthesis by adipose cells and higher levels of TNF in serum. Gastrointestinal cancer patients also have greater insulin resistance than non-cancer patients, and the degree of insulin resistance is highly correlated with TNF levels. TNF appears to be responsible for inhibiting the tyrosine phosphorylase activity of the IR, although the exact mechanism of this effect is not known. While the effect of physical activity on this potential mechanism has not been tested, an exercise-stimulated increase in expression of soluble TNF-r1 (sTNF-r1) and sTNF-r2 should compete for circulating TNF and attenuate TNF-associated effects (see FIG. 1).

FIG. 1 illustrates the effect of cachexia and physical exercise on the GLUT4 activation pathways. Inflammation-induced TNF blocks activity of the insulin-activated IR. Physical exercise-suppression of inflammatory responses (induced IL-10, IL-Ira, and sTNF-r 1,2) may block the synthesis/release of TNF thereby attenuating the cachexia-associated production of insulin resistance. Activation of the GLUT4 pathways through enhanced NO, cGMP, 5′-AMP-PK activity, GLUT4 synthesis, PI-3 kinase activity, and GLUT4 translocation by physical exercise will enhance insulin sensitivity and possibly prevent or reverse cachexia-associated insulin resistance. Physical activity is well known for enhancing insulin sensitivity in skeletal muscle under non-cachexic/non-inflammatory circumstances.

Although all the mechanisms responsible for exercise-enhanced insulin sensitivity in skeletal muscle have not been completely delineated, enhanced translocation of the GLUT4 protein to the cell membrane and enhanced expression of GLUT4 protein were among the first adaptations described. Regulation of GLUT4 translocation to the cell membrane is extremely complex with multiple pathways of insulin-independent stimulation being described including: nitric oxide/cGMP-stimulated translocation which is independent of both the Ca++/contraction and PI-3-kinase mediated translocation, an increase in 5′-AMP activated protein kinase activity, and a bradykinin-dependent mechanism. Physical activity and physical exercise also increase expression of the GLUT4 protein itself. All of these changes enhance the number of GLUT4 transporters which migrate to the cell membrane in response to a given dose of insulin and subsequently enhance the transport of glucose and amino acids (see FIG. 1). Equally important are the alterations in the insulin-mediated signal transduction pathway that are now known to occur.

Briefly, insulin binds to the insulin receptor (IR) which phosphorylates the insulin receptor substrate (IRS-1 & IRS-2). Phosphorylated IRS attracts phospatidylinositol-3 kinase (PI-3-K) and P85, which, when P85 complexes with PI-3-K, activates the PI-3-K, producing phosphatidylinositol 3,4,5 triphosphate (PI-3,4,5-TP) from PI-4,5 bisphosphate. PI-3,4,5-TP attracts and activates a variety of kinases including protein kinase B (PKB) which is most likely involved in activating GLUT4 to translocate from intracellular vesicles to the cellular membrane (see FIG. 1). Recent work demonstrates that expression of both PI-3-K and ras is significantly enhanced by repeated swimming activity while PI-3-K activity is significantly elevated within one day of running activity. These alterations in the insulin receptor-mediated signaling pathway also increase cell sensitivity to insulin. Indeed, as previously mentioned, physical exercise has been observed to reverse some of the muscle wasting in tumor-bearing rats, an effect resulting in part from enhanced insulin sensitivity. These potential physical exercise-induced effects would be very important in a cachexic patient because they could contribute to a reversal (or prevention) of the observed insulin-resistance in cachexia.

Glucose transport through non-insulin mediated pathways also is important to maintaining energy supplies within muscle. GLUT1 and GLUT3 transporters in skeletal muscle are vital to basal glucose uptake and expression of GLUT 3 transporters has been recently documented to be markedly lower in insulin resistant patients. The importance of this is that regulation of synthesis of glucose transporters (and other glycolysis-related enzymes) is through the hypoxia-inducible factor (HIF-1); transactivation of which is mediated by the tyrosine kinases of the MAPK pathway of transcription activation. Because exercise activates MAPK pathways through enhanced reactive oxygen species and calcium (see below), an enhanced expression of insulin-independent glucose transporters also will contribute to enhancing glucose uptake and attenuation of wasting.

Increasing transport of glucose and amino acids through enhanced insulin sensitivity would provide partial reversal of the muscle wasting process by increasing substrate concentration. Enhancing rates of protein synthesis through activating transcription and translation also would be very important to prevent the wasting process. Insulin, growth hormone (GH), and physical exercise enhance rates of protein synthesis with the mitogen activated protein kinase pathway (MAPK pathway: Ras-Raf-MEK-ERK) being the common denominator.

Insulin is known to be involved in stimulating enhanced rates of protein synthesis in skeletal muscle following weight lifting activity.

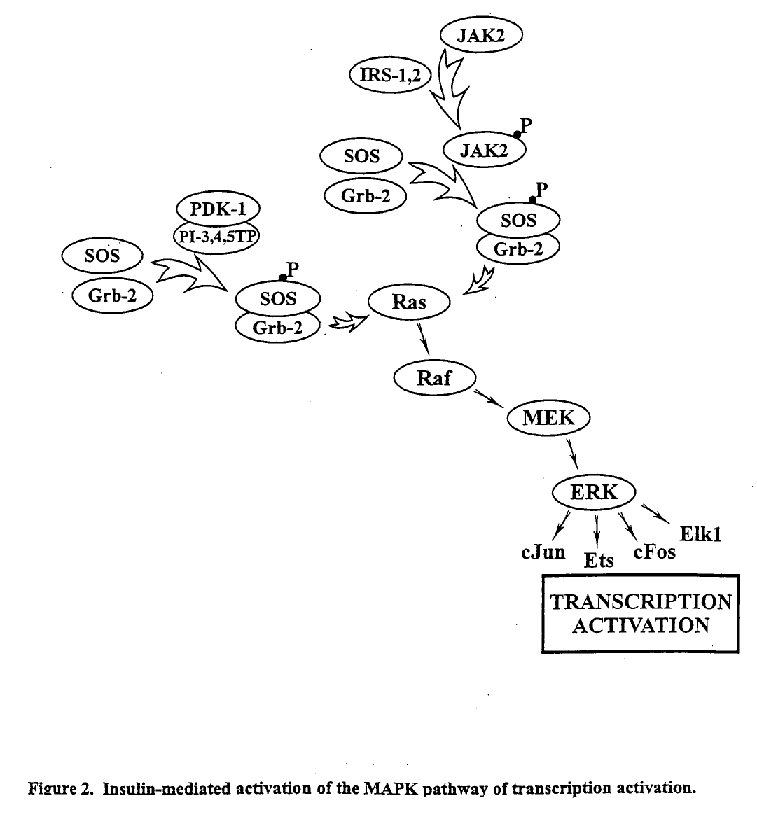

FIG. 2 illustrates the insulin-mediated activation of the MAPK pathway of transcription activation. Both the activated IRS-1,2 (through activated Jak-2) and the PDK-1:PI-3,4,5TP complexes can activate the SOS:Grb-2 complex which in turn activates Ras of the MAPK pathway, resulting in enhanced protein synthesis.

The effect of insulin on the MAPK signal transduction pathways may contribute to this enhanced protein synthesis. The phosphoinositide dependent kinase-1 (PDK-1)/PI-3,4,5 TP complex, which phosphorylates PKB to activate GLUT4 translocation, also activates the SOS/Grb-2 complex which then activates Ras and the MAPK pathway (see FIG. 2), leading to the phosphorylation (activation) of a variety of transcription activators. The active IRS also can phosphorylate Jak-2 (Janus Kinase-2) which subsequently activates the SOS/Grb-2 complex.

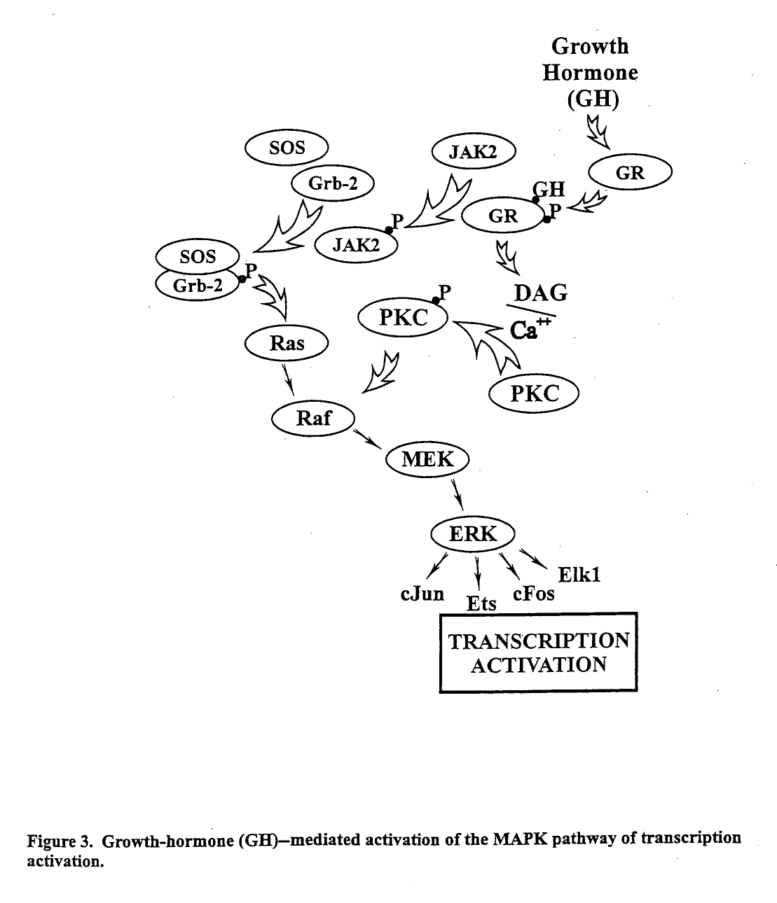

The MAPK pathway plays a central role in the growth hormone mechanism of action. GH is well known to enhance rates of protein synthesis, and it appears to work, in part, through a protein kinase C (PKC)-mediated (PKC is activated by diacylglycerol/calcium: DAG/Ca⁺⁺) enhanced activation and expression of the Jun and Fos transcription activators (see FIG. 3).

FIG. 3 illustrates the growth hormone (GH)-mediated activation of the MAPK pathway of transcription activation. The activated growth-hormone receptor (GR) activates JAK2 which mediates the activation of Ras via the SOS:Grb-2 complex. Activated GR also stimulates the release of Ca++into the cytosol (via DAG) which activates PKC. PKC in turn activates Raf of the MAPK pathway; activation of both Ras and Raf results in enhanced protein synthesis.

GH stimulated expression offos and transcription activation mediated by the fos promoter are inhibited by a MEK inhibitor (MEK: mitogen activated protein kinase/extracellular regulated kinase; a component of the MAPK pathway) as are other serum response element (SRE) regulated genes. In addition to activating PKC, the activated GR also can activate Jak-2, providing two separate mechanisms through which GH enhances activity of the MAPK pathway resulting in increased rates of protein synthesis. Because the MAPK pathway is central to the mechanism of enhanced protein synthesis, one hypothesis is that a physical exercise-induced increase in insulin sensitivity would mediate an attenuation of cachexia-associated muscle wasting. In addition to this possibility, exercise also appears to activate the MAPK pathway independently of insulin.

While one bout of 30 minutes of moderate activity does not affect the MAPK pathway, 60 minutes of activity enhances this activity. Repeated physical activity over several weeks also results in an induced MAPK activity as well, indicating that the ability for physical exercise to activate the phosphorylating activity of this pathway is not diminished with repeated bouts of activity. In addition to activating MAPK activity, physical activity also enhances the expression of jun and fos and the fos/jun families of transcription activators. These effects are the same as those observed with GH and indicate that events associated with muscle contraction probably stimulate the same signal transduction pathways. Because PKC is involved in GH-mediated expression of fos, it stands to reason that flooding a muscle cell with calcium to stimulate contraction also would stimulate PKC enzymes, leading to enhanced transcription activation. In addition to greatly enhanced Ca++levels, production of reactive oxygen species (ROS) in muscle cells also is greatly enhanced by exercise.

The enhanced production of ROS by exercise is important because ROS are involved as secondary messengers in the activation of PKC by signaling pathways which transduce signals from the cell membrane to the nucleus. ROS also are able to directly activate PKC and inhibitors of PKC, such as calphostin C, prevent oxidant-mediated induction of fos expression. The DNA binding activity of Jun also is enhanced following activation of PKC indicating that ROS mediated activation of PKC results in activation of the MAPK pathway.

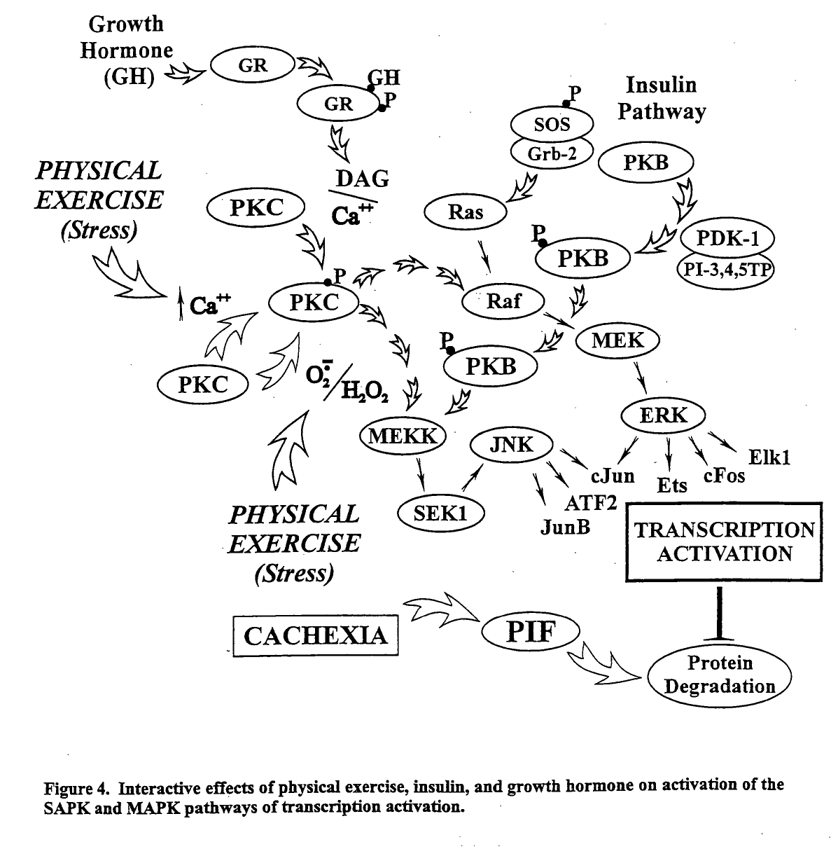

In addition to the MAPK pathway, the c-Jun N-terminal Kinase pathway (JNK-MAPKs—also known as the stress activated pathway, or SAPK: MEKK-SEKI-JNK) can be activated by a variety of signals including insulin and growth hormone resulting in enhanced transcription activation. PKB (insulin pathway) can directly activate MEKK while ROS and Ca⁺⁺ are associated with activating PKC activity which in turn results in the activation of the JNK-MAPK pathway. Because both ROS and Ca⁺⁺ are induced during cellular and exercise stress, ROS and Ca⁺⁺ are the intracellular signals responsible for the stress-associated activation of both the MAPK and JNK-MAPK pathways (see FIG. 4) and anything which transiently enhances their cellular content would have the same effect as acute exercise.

FIG. 4 illustrates the interactive effects of physical exercise, insulin, and growth hormone on activation of the SAPK and MAPK pathways of transcription activation. PKC is activated by physical exercise through enhanced Ca⁺⁺ and ROS as well as by growth hormone through DAG/Ca⁺⁺. PKC in turn activates the SAPK and MAPK pathways by activating MEKK and Raf, respectively. Insulin activates PKB and the SOS:Grb-2 complex which in turn activate the SAPK and MAPK pathways by activating MEKK and Ras, respectively. By enhancing insulin sensitivity and activating SAPK and MAPK independently of insulin and growth hormone, physical exercise should greatly enhance rates of protein synthesis beyond that expected by insulin or GH alone; resulting in an attenuation or prevention of cachexia-associated muscle wasting and fatigue.

The ability of a muscle contraction-mediated increase in cellular ROS and Ca⁺⁺ to activate MAPK and JNK-MAPK, resulting in increased protein synthesis, is significant because it would explain how hard or prolonged muscle contractions initiate an increase in protein synthesis beyond that related to growth hormone alone. Thus, repeated physical activity appears to activate mechanisms which have the potential to reverse (or prevent) the cachexia often seen in cancer patients. Exercise-induced increases in insulin sensitivity and in protein synthesis would counteract the TNF-mediated decrease in insulin sensitivity and the PIF-induced (muscle) proteolysis associated with cachexia.

In addition to affecting mechanisms directly associated with muscle wasting, physical exercise may provide other benefits to the cancer patient by enhancing protection from the side effects of chemo- and radiation-therapy as well as attenuating carcinogenesis. ROS production from cancer-associated activation of inflammatory cells, from radiation therapy, and from metabolism of therapeutic drugs may play a large role in the debilitating effects of cancer and cancer therapy. ROS can initiate an inflammatory response which can then lead to the sequelae of events resulting in cachexia. On the other hand, physical exercise is known for its ability to enhance antioxidant enzyme activity. Repeated physical activity enhances the mitochondrial Mn-superoxide dismutase (Mn-SOD) and glutathione peroxidase (GPX) activities in skeletal muscle while Mn-SOD (or cytoplasmic+mitochondrial SOD) and catalase (CAT) are induced in lung and diaphragm. These antioxidant enzymes are very important in protecting the cell from damage associated with elevated ROS and markers of ROS damage are reduced following physical exercise. Enhanced antioxidant function induced by exercise could play a role in the exercise-induced attenuation of cachexic symptoms and muscle wasting.

ROS are intimately involved in the cytotoxic effects of chemotherapy, radiation therapy, and inflammatory-associated damage. ROS also are implicated as part of the mechanism for many cancers; including those associated with the tobacco-specific nitrosamine: 4-(methyInitrosamino)-1-(3-pyridyl)-1-butanone (NNK). When the rate of cell division exceeds the rate at which ROS-mediated DNA damage can be repaired, then the rate at which mutations form is increased, and the risk for cancer increases. The rate of ROS-mediated DNA-adduct formation can be as great as 10,000 lesions per cell each day from endogenous sources. 8-Hydroxy-deoxyguanosine (8-OH-dG), a hydroxyl radical-caused DNA adduct, is elevated in mice treated with NNK while the consumption of (−)-epigallocatechin gallate (EGCG), a potent antioxidant found in green tea, significantly lowers levels of 8-OH-dG in lung DNA and decreases incidence of cancer in NNK treated animals. Thus, the enhanced antioxidant capacity due to repeated exercise may well contribute to the observed reduced risk for cancer by physical exercise or participation in athletics.

Because significant inverse correlations (greater activity produces lower risk) exist between activities of the phase II enzymes GST and UDP-GT and the incidence of cancer, induction of these enzymes, which inactivate carcinogens and toxins derived from metabolism of chemicals and pharmaceuticals, also would contribute to cancer protection. Published data from Applicant's lab has indicated that, in addition to inducing antioxidant enzyme activity in lung and liver, repeated exercise in rats also enhances UDP-GT activity in lung and liver. These combined effects should reduce risk for carcinogenesis as well as provide enhanced protection from the metabolic production of ROS and reactive chemical intermediates resulting from chemotherapy.

Recent evidence indicates that enhanced antioxidant capacity (Cu/Zn-SOD and Mn-SOD) of some tumor cells decreases tumor cell growth in vitro. These results raise the possibility that physical exercise, in addition to attenuating carcinogenesis, also may reduce cancer-cell growth (through a non-endocrine mechanism). This would happen, however, only if the tumor cells respond to exercise in a similar manner as normal tissues.

Exactly how the regulation of the phase II enzymes may be affected by exercise has not been tested directly; however, an interesting “cross-talk” situation exists between transcription activators for antioxidant enzymes, phase II enzymes, and enhanced protein synthesis. The transcription activator AP-1 0un/fos) is involved in enhancing transcription of UDP-GT, GST, and cytochrome P450 1A1 as well as proteins associated with transcription and translation, while NFicp is involved in the transcription activation of Mn-SOD and elk-I (activated via MAPK pathway) is an activator for Cu/Zn SOD.

NF-κβ, when activated, rapidly enters the nucleus where it initiates transcription of a variety of genes including mitochondrial Mn-SOD. NF-κβ is usually activated following stimulation of the cell by the cytokines interleukin-1β or tumor necrosis factor a while the disassociation of the inhibitory subunit from NF-κβ which activates this transcription factor is actually mediated by the secondary messenger H202. Interestingly enough, as described above, ROS also can activate jun/fos as well as PKCs, illustrating the potentially central role of exercise-induced production of ROS in activating a cellular response which ultimately enhances rates of protein synthesis and increases both antioxidant and phase II enzyme activities. Each of these mechanisms contributes to a decreased risk for carcinogenesis and growth-inhibition of metastases.

The apparent paradox of an exercise-associated increase in ROS production being beneficial might be explained by the duration and intensity of the increase. Physical activities which produce lactic acid and fatigue are by necessity of short duration. A transient (one hour or less) and intermittent (3 times each week on non-consecutive days to no more than three times each day) increase in ROS production due to physical exercise may be sufficient to activate the various transcription enhancers described above to a sufficient degree in order to obtain beneficial (protective) outcomes without suffering the damaging effects of a chronically enhanced ROS production (such as that due to inflammation). In addition to enhanced ROS production, this distinction in the duration of activation is important because many of these exercise/stress-responsive transcription activators (as discussed above) are known oncogenes, which, when constituently active, are high risk factors in cancer development.

Example of the Method Using Exercise as the Test Protocol.

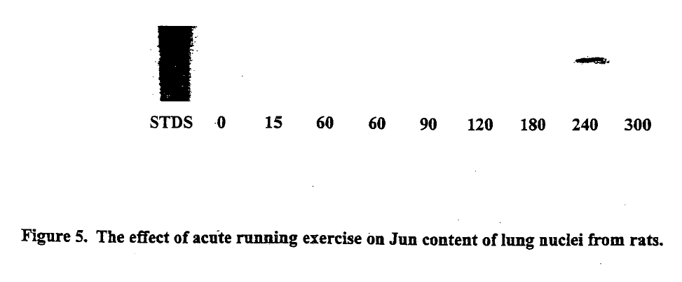

FIG. 5 illustrates the effect of acute running exercise on Jun content of lung nuclei from rats. Briefly, rats were familiarized with a rodent treadmill on four separate days over a period of two weeks and then forced to run for 60 minutes at a speed of 27 m/min (a moderately hard workload for untrained rats). Three animals were killed at each time point: 0, 15, 30, 60, 90, 120, 180, 240, and 300 minutes after the start of the exercise. Lungs from the three animals at each time point were pooled and nuclei prepared by Dounce homogenization and differential centrifugation. Nuclear proteins were analyzed by western blot as previously described using anti cJun/AP1 monoclonal antibody (Ab-3, Oncogene Research Products). The blot was digitized, converted to grayscale, and the 35-45 kDa region of the blot which included the immunoreactive protein was printed using an HP Photosmart 1215 printer at highest resolution. Immuno-reactive Jun protein appeared in the lung nuclei 4 hours after the start of the exercise and one hour later jun content was again below detectable levels. This indicates that a 60 minute bout of running exercise is sufficient to enhance jun content of lung nuclei but that this effect is of relatively short duration. Based on these results a transient exercise/stress-mediated activation of the MAPK and/or JNK-MAPK pathways through enhanced Ca⁺⁺ and ROS is responsible for the enhanced activity of antioxidant enzymes and phase II enzymes previously observed. Because skeletal muscle is far more metabolically active than lung during exercise the degree of an exercise-induced activation of AP-1 in muscle would be greater than that of lung, yet it is still a transient increase in gene activation.

Any treatment which enhances the endogenous activation of any of these signal transduction or gene pathways will enhance endogenous activity of antioxidant and phase II enzymes, enhance insulin sensitivity, and increase rates of protein synthesis in skeletal muscle. Acute exercise activates these pathways through transient increases in cellular content of reactive oxygen species (H₂O₂) and calcium. These same pathways also are activated by transient increases in diacylglycerol or phosphatidylinositol metabolites. Based on these observations, any treatment which activates these same pathways also will result in these same benefits.

Accordingly, Applicant conceived of the method of the present invention which builds on these observations to create a method for screening agents or compounds for use as drugs which will provide similar benefits as described above for exercise. If the tested agent or compound activates one of these pathways, then it should provide these benefits and be useful for treatment and prevention of cachexia and other wasting disorders, cancer, atherosclerosis, heart disease, autoimmune disease, chronic inflammatory disease, alcoholic hepatitis, nonalcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities.

Further, the ability of the specific agents DAG, PI-metabolites, and H₂O₂ to transiently activate the gene pathways has been clearly demonstrated in cell culture experiments as already discussed above. The use of the calcium ionophore A23817 and the endoplasmic calcium ATPase inhibitor thapsigargin in cultured keratinocytes clearly demonstrates that a transient increase in cytosolic calcium stimulated by either agent leads to a transient increase in cytosolic H₂O₂ which in turn leads to an oxidized state which lasts for no more than 20 minutes. The oxidized state enhances activation of the apoptosis signaling kinase and MAPK pathways to initiate increased rates of protein synthesis and growth.

It will be understood that the embodiments and examples of the present invention, which have been described herein, are illustrative of some of the applications of the principles of the present invention. Numerous modifications may be made by those skilled in the art without departing from the spirit and scope of the invention.

What is claimed as new and desired to be protected by Letters Patent is set forth in the appended claims.

I claim:

1. A method of screening agents to determine if such agents are useful for the prevention and treatment of disorders selected from the group consisting of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities, said method comprising the steps of:
treating cells of a living organism with a putative agent; and
measuring transient activation of a stress-response pathway,
wherein said transient activation indicates said putative agent is useful for prevention or treatment of said disorders.

2. The method of claim 1, wherein said stress-response pathway is selected from the group consisting of mitogen activated protein kinase (MAPK) pathways of transcription activation, stress activated protein kinase pathways of transcription activation, hypoxia inducible factor (HIF) pathways of transcription activation, nuclear factor kappa-beta (Nf-icp) pathways of transcription activation, and insulin-associated GLUT4 activation or translocation pathways.

3. The method of claim 1, wherein said transient activation includes transient activation of any of the genes or signaling molecules within said pathways.

4. The method of claim 1, wherein said step of measuring transient activation is performed using a methodology selected from the group consisting of western blot, dot-blot, ELISA, immunocytochemistry, immunohistochemistry, mass-spectroscopy, multiplexed proteomics technology, antibody microarray, or truncated protein analysis to determine enhanced content of gene product(s) in cells, nuclei, or enhanced appearance of gene products within nuclear-protein extracts; phosphorylation-based assays to determine pbosphorylation state of any of the gene products, phospholipids, diacylglycerol products or other signaling molecules in said gene or stress-response pathways; gel-shift assays to determine enhanced sequence-specific DNA-binding of the various genes involved in the above-mentioned pathways (such as, but not limited to, AP-1); any reporter-gene assays to determine gene activation; any RT-PCR-based assays (including real-time RT-PCR, in situ hybridization, quantitative and semi-quantitative RT-PCR, and competitive quantitative RT-PCR) to quantify content of mRNA coding for the gene product(s) in question; or any of the RNA- or protein-array or microarray methodologies available to determine gene activation.

5. The method of claim 1, wherein said transient activation of a stress-response pathway indicates that said putative agent is a source of an agent for treatment of said disorders.

6. The method of claim 1, wherein said transient activation is longer than approximately 1 minute and is no longer apparent 12 hours after initial activation.

7. The method of claim 6, wherein said transient activation occurs not more than three times in one day and not less than once each day for three non-consecutive days.

8. The method of claim 1, wherein said transient activation is produced by a treatment modality selected from the group consisting of modifying dosing regimens, modifying dosing apparatus, modifying dosing formulations, modifying pharmacokinetic properties of said agent, modifying other properties of said agent, modifying vehicle of said agent, and modifying delivery apparatus of said agent.

9. The method of claim 1 wherein said cells of a living organism are mammalian cells.

10. The method of claim 9, wherein said mammalian cells are selected from the group consisting of human cells and primate cells.

11. The method of claim 1, wherein said transient activation of a stress-response pathway is selected from the group consisting of an activation of any of a signaling processes which may result in increased synthesis of any of gene products associated with the stress-response pathways, increased nuclear content of any of gene products associated with the stress-response pathways, and increased cellular content of any of gene products associated with the stress-response pathways.

12. A method of screening agents to determine if such agents are useful for the prevention or treatment of wasting disorders associated with a disorder selected from the group consisting of cachexia and other wasting disorders, cancer, atherosclerosis, heart disease, autoimmune disease, chronic inflammatory disease, alcoholic hepatitis, non-alcoholic hepatitis, rheumatoid arthritis, osteoarthritis, type II diabetes, insulin insensitivity, Parkinson's disease, Alzheimer's disease, and any other condition caused or mediated by chronic oxygen radical damage or by chronic chemical toxicities, said method comprising the steps of:
treating living organisms with a putative agent; measuring transient activation of a stress-response pathway,
wherein said transient activation indicates said putative agent is useful for prevention or treatment of said wasting disorders.

13. The method of claim 12, wherein said stress-response pathway is selected from the group consisting of mitogen activated protein kinase (MAPK) pathways of transcription activation, stress activated protein kinase pathways of transcription activation, hypoxia inducible factor (HIF) pathways of transcription activation, nuclear factor kappa-beta (Nf-κβ) pathways of transcription activation, and insulin-associated GLUT4 activation or translocation pathways.

14. The method of claim 12, wherein said transient activation includes transient activation of any of the genes or signaling molecules within these pathways.

15. The method of claim 12, wherein said step of measuring transient activation is done using a methodology selected from the group consisting of western blot, dot-blot, ELISA, immunocytochemistry, immunohistochemistry, mass-spectroscopy, multiplexed proteomics technology, antibody microarray, or truncated protein analyses to determine enhanced content of gene product(s) in cells, nuclei, or enhanced appearance of gene products within nuclear-protein extracts; phosphorylation-based assays to determine phosphorylation state of any of the gene products, phospholipids, diacylglycerol products or other signaling molecules in said gene or stress-response pathways; gel-shift assays to determine enhanced sequence-specific DNA-binding of the various genes involved in the above-mentioned pathways (such as, but not limited to, AP-1); any reporter-gene assays to determine gene activation; any RT-PCR-based assays (including real-time RT-PCR, in situ hybridization, quantitative and semi-quantitative RT-PCR, and competitive quantitative RT-PCR) to quantify content of mRNA coding for the gene product(s) in question; or any of the RNA- or protein-array or microarray methodologies available to determine gene activation.

16. The method of claim 12, wherein said transient activation is longer than approximately 1 minute and is no longer apparent 12 hours after initial activation.

17. The method of claim 16, wherein said transient activation occurs not more than three times in one day for seven days and not less than once each day for three consecutive days.

18. The method of claim 12, wherein said transient activation is produced by a treatment modality selected from the group consisting of modifying dosing regimens, modifying dosing apparatus, modifying dosing formulations, modifying pharmacokinetic properties of said agent, modifying other properties of said agent, modifying vehicle of said agent, and modifying delivery apparatus of said agent.

19. The method of claim 12 wherein said living organisms are mammals.

20. The method of claim 19, wherein said mammals are selected from the group consisting of humans and primates.

21. The method of claim 19, wherein said treating living mammals includes treating cultured tissues from said mammals.

22. The method of claim 21, wherein said cultured tissues are from a human.

23. The method of claim 12, wherein said transient activation of a stress-response pathway is selected from the group consisting of an activation of any of a signaling processes which may result in increased synthesis of any of gene products associated with the stress-response pathways, increased nuclear content of any of gene products associated with the stress-response pathways, and increased cellular content of any of gene products associated with the stress-response pathways.
